(19) United States
(12) Patent Application Publication
(10) Pub. No.: US 2020/0037896 A1
(43) Pub. Date: Feb. 6, 2020

(54) OPTICAL FLOW ANALYSIS METHOD AND DETECTION DEVICE

(71) Applicants: Guillermo Aguilar-Mendoza, Corona, CA (US); Nami Davoodzadeh, Riverside, CA (US); AmirHessam Aminfar, Riverside, CA (US)

(72) Inventors: Guillermo Aguilar-Mendoza, Corona, CA (US); Nami Davoodzadeh, Riverside, CA (US); AmirHessam Aminfar, Riverside, CA (US)

(21) Appl. No.: 16/528,406

(22) Filed: Jul. 31, 2019

Related U.S. Application Data
(60) Provisional application No. 62/714,569, filed on Aug. 3, 2018.

(51) Int. Cl. A61B 5/026; A61B 5/00

(52) U.S. Cl. CPC A61B 5/0261; G01N 2021/479; A61B 5/489

(57) ABSTRACT

A method of optical flow analysis and a detection device using the analysis method is shown. Examples shown include imaging using a fluid mechanic approach to visualizing flow, for example, in organic tissue. Example shown use apparent displacement in laser speckle patterns.

### RELATED APPLICATION

This application claims priority to U.S. Provisional Patent Application No. 62/714,569 entitled “Laser Speckle Imaging of Blood Flow Using Optical-Flow Image Analysis,” filed on Aug. 3, 2018, which is incorporated herein by reference in its entirety.

### STATEMENT OF GOVERNMENT GRANT

The U.S. Government has certain rights in this invention pursuant to National Science Foundation (NSF) Award Nos. 1707190, 1547014, and 1545852.

### TECHNICAL FIELD

This invention relates to flow detection and methods. In one example, this invention relates to optical flow detection in biological tissue.

### BACKGROUND

Since of its introduction in 1980s, laser speckle imaging has become a powerful tool in flow imaging. Its high performance and low cost made it one of the preferable imaging methods. Initially, speckle contrast measurements were the main algorithm for analyzing laser speckle images in biological flows. Improved imaging of flowing media, such as blood flow in biological tissue, are desired.

### BRIEF DESCRIPTION OF THE DRAWINGS

FIG. 1 shows photographs acquired using a number of imaging techniques according to an example of the invention.

FIG. 2 shows photographs acquired by additional imaging techniques according to an example of the invention.

FIG. 3 shows photographs using different numbers of data frames acquired by additional imaging techniques according to an example of the invention.

FIG. 4 shows photographs using different filters acquired by additional imaging techniques according to an example of the invention.

FIG. 5 shows blood vessel photographs acquired by additional imaging techniques according to an example of the invention.

FIG. 6 shows data for apparent displacement for different calculation methods according to an example of the invention.

FIG. 7 shows an overview of an imaging technique for organic tissue according to an example of the invention.

FIG. 8 shows a simplified graphical illustration of displacement calculations according to an example of the invention.

FIG. 9 shows a more detailed graphical illustration of displacement calculations according to an example of the invention.

FIG. 10 shows comparison imaging of four different imaging techniques according to an example of the invention.

FIG. 11 shows an image with tubular flow estimation according to an example of the invention.

FIG. 12 shows a flow diagram of a method of imaging according to an example of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

Since of its introduction in 1980s, laser speckle imaging has become a powerful tool in flow imaging. Its high performance and low cost made it one of the preferable imaging methods. Initially, speckle contrast measurements were the main algorithm for analyzing laser speckle images in biological flows. Speckle contrast measurements, also referred as Laser Speckle Contrast Imaging (LSI), use statistical properties of speckle patterns to create mapped image of the blood vessels. In this communication, a new method named Laser Speckle Optical Flow Imaging (LSOFI) is introduced. This method uses the optical flow algorithms to calculate the apparent motion of laser speckle patterns. The differences in the apparent motion of speckle patterns are used to identify the blood vessels from surrounding tissue. LSOFI provides better spatial and temporal resolution compared to reported LSI methods. This higher spatial resolution enables LSOFI to be used for autonomous blood vessels detection. Furthermore, Graphics Processing Unit (GPU) based LSOFI can be used for quasi real time imaging.

Imaging blood flow in the tissue is of major importance in the clinical environment. Over recent decades, several techniques have been developed for imaging tissue perfusion. Most of these techniques exploit the interference pattern generated from diffusely backscattered light from the skin. Currently, laser speckle contrast techniques are gaining interest. Laser speckle contrast techniques are based on the spatial and temporal statistics of the speckle pattern. The motion of particles in the illuminated medium causes fluctuations in the speckle pattern on the detector.

These intensity fluctuations blur the image and reduce the contrast to an extent that is related to the speed of the illuminated objects, such as moving red blood cells. Several researchers used the principle of speckle contrast to develop techniques for measuring skin perfusion. However, the special and temporal resolution have not changed significantly.

An infrared diffused laser aluminates a sample tissue. Then, speckle pattern is acquired through a CMOS camera associated with a 10× magnifier lens. In the presented laser speckle imaging method, optical flow algorithm is applied to a speckle image in order to map the flow perfusion. Optical flow (OF) generally is used to computes the displacement between two consecutive frames. In our experiments the algorithm captures these movements and computes a magnitude for displacement. By evaluation of the magnitudes the blood perfusion is mapped.

We used optical flow algorithms including horn-chunk and Lucas Kannade. In our experiments random speckle noise movement is generated by various physical phenomena, Hence, when OF algorithm is applied to a speckle image, the algorithm captures these movements and computes a magnitude for displacement. It can be seen from the images that, where there is a steady state flow, the magnitude of displacement decreases, and the places that there isn't any flow, we have higher displacement magnitude. If we average all the displacement magnitude in time, the resolution of the image increases.

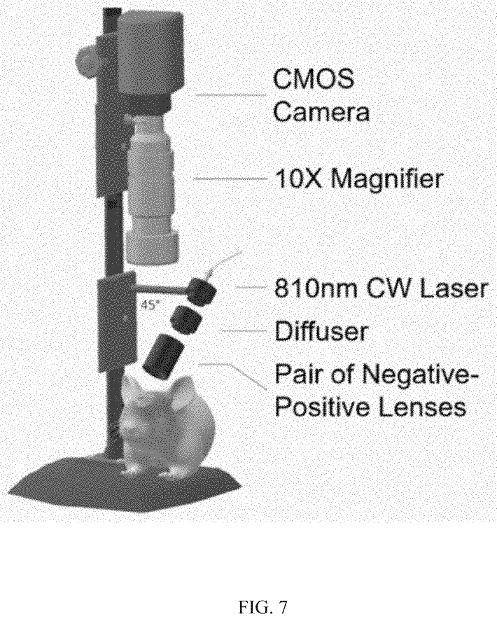

A visual explanation is provided in FIGS. 7-11. By using a two-frame comparison analysis, higher temporal resolution (−10 ms) is achieved in comparison with the conventional contrast analysis approaches. In comparison with high temporal resolution speckle image analysis methods, using displacement magnitudes instead of pixels contrasts for mapping the flow, provides a higher special resolution. Low CPU load, the analysis method does not require a high processor capacity which enables the imaging method to be used in microcomputers and compact and cheap devices.

FIG. 7 shows how light from an IR laser diode was collimated to illuminate the exposed brain surface at an angle of 0° off the normal direction. A microscope mount (10×) above the cranial window relayed the brain surface image onto a monochromatic CMOS camera. The exposure duration (T) was set to 10 ms and the acquired image sequence was transferred to a PC. We used optical flow algorithms including horn-chunk and LucasKannade, when OF algorithm is applied to a speckle image, the algorithm captures these movements and computes a magnitude for displacement.

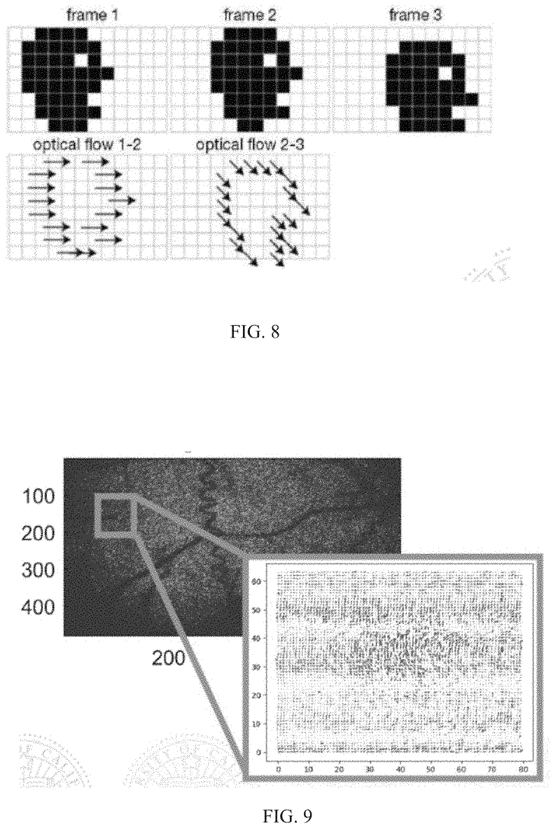

FIGS. 8 and 9 show how optical flow (OF) is used to compute the displacement between two consecutive frames. In our experiments random speckle noise movement is generated by various physical phenomena.

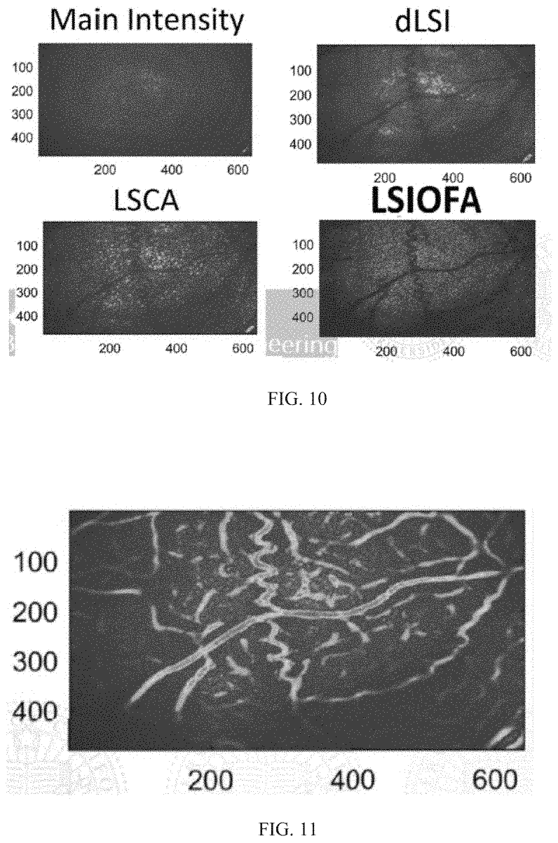

FIGS. 10 and 11 show a comparison between the contrast analysis (LSCA), dynamic speckle analysis (dLSI), and our optical-flow image analysis (LSIOF) method using a sample cerebral mouse brain speckle images

### INTRODUCTION

Because of the importance of accurate blood flow visualization, various techniques for flow imaging have been proposed in medical fields such as ophthalmology, dermatology, endoscopy, and internal medicine. Some of these techniques are non-invasive and include methods such as Orthogonal Polarization Spectral (OPS) imaging, side Stream Dark Field (SDF), Laser Doppler Perfusion Imaging (LDPI) and laser speckle methods, such as Laser Speckle Contrast Imaging (LSCI). Among all these methods, LSCI is gaining interest because of its simplicity and ease of use.

Laser speckle effect occurs when a coherent light, such as a laser beam, illuminates a rough diffuse surface, thereby producing random interference effects. This effect is visualized by a granular pattern consisting of dark and bright spots. When the speckle pattern is generated on a moving object such as blood vessels, the blood flow causes fluctuations in the speckle pattern on the detector. In fluid mechanics, the phenomenon where an optical wave propagating through a medium experience irradiance (intensity) fluctuations, is referred to as optical turbulence. In LSCI, blood flow causes blurriness of image pixels, which leads to scintillation of the intensity.

Here we define scintillation index as:

$$S = \frac{〈 {I}^{2} 〉 - {〈 1 〉}^{2}}{{〈 I 〉}^{2}} \qquad ( 1 )$$

where I denotes intensity of the optical wave, and < > denotes ensembled average or a long time average. Over the years various algorithms and methods have been developed to calculate both temporal and spatial fluctuations of speckle patterns. These methods measure the blurriness of image pixels, which is generally centered around speckle contrast defined as:

$$κ = \frac{σ}{〈 I 〉} = \frac{\sqrt{〈 {I}^{2} 〉 - {〈 I 〉}^{2}}}{〈 I 〉} \qquad ( 2 )$$

Obviously, the speckle contrast, κ, is the same as the square root of the scintillation index defined in Eq. (1). When there is little or no movement in the speckle patterns, “fully developed” patterns will cause the speckle contrast (Eq. (2)) to be equal to unity. When there is movement in the object, the speckle pattern blurs, and the standard deviation of the intensity will be smaller than the mean intensity, thereby reducing speckle contrast. Assuming that the motion of the scattering areas of the flow is random and these random motions will decorrelate in time, the speckle contrast could be correlated to blood flow velocity. The speckle contrast is furthermore related to normalized electric field autocorrelation function g₁(r, τ) as

$$κ = \frac{2 β}{T} {\int }_{0}^{T} \vert {g}_{1} {\vert}^{2} ( 1 - \frac{τ}{T} ) d τ \qquad ( 3 )$$

where β is a constant which account for different optical modes in measurement. Here τ is time and T is the averaging period. Typically, g₁ is hard to measure and therefore the intensity correlation function g₂(τ) is measured instead. Functions g₂(τ) and g₁(τ) are correlated through Siegert relation as Fercher and Briers (1981) assumed that β=1 and the measured intensity of speckles is integrated over time. Doing so, they came up with the first speckle model described as

$$κ = \sqrt{\frac{{τ}_{c}}{2 T} ( 1 - exp ( - \frac{2 T}{{τ}_{c}} ) )} \qquad ( 4 )$$

where τ_{c} is the decorrelation time and is linked to decorrelation velocity v_{c} defined as

$${v}_{c} = \frac{λ}{2 π {τ}_{c}} \qquad ( 5 )$$

where λ is the laser wavelength. Eq. (4) is one of the early attempts to correlate the speckle decorrelation time to speckle contrast κ. The speckle contrast, κ, can be used to create a mapped image. In this image values of speckle contrast, κ, are different for the blood flow and surrounding tissue. This difference enables identifying the blood vessels in the image. Eq. (4) has been evolving over the years. The developments have been in various methods for measurement of κ and decorrelation time τ_{c}. The newer methodologies take into account various aspects of laser speckle patterns such as the presence of static layer on top of the blood vessels. Theoretical efforts were established to quantify the impact of speckle size and sampling windows on the statistics of laser speckle. Moreover, studies have been done to improve laser speckle imaging algorithms. These improvements enable real-time blood flow visualization. Since the normalized electric field autocorrelation function g₁(τ) is related to the mean square displacement <|Δr²|> of the speckle patterns; it can be assumed that in the blood vessels, <|Δr²|> is caused by the flow. Therefore, instead of using statistical behavior of laser speckle patterns, a physics based model of the blood flow can be deployed for flow visualization. In the most general form, Cauchy momentum equation can be used to describe the blood flow. However, since in many physiological conditions blood is assumed to be a Newtonian fluid, The Navier-Stokes equation can be used to describe the motion of the flow. The blood flow is considered pulsatile viscous flow. Assuming vessels as rigid tubes, simplified Navier-Stokes can be solved in different forms including:

$$u ( r , t ) = Real \{ {\sum }_{n = 0}^{N} \frac{{i•P}_{s}}{ρ•ω} ( 1 - \frac{{J}_{0} ( {n}^{\frac{1}{2}} {W•i}^{\frac{3}{2}} • \frac{r}{R} )}{{J}_{0} ( {n}^{\frac{1}{2}} {W•i}^{\frac{3}{2}} )} ) ) {• e}^{inux} \} \qquad ( 6 )$$

where u(r, t) is the radial component of the flow, W=R·ρω/μ is nondimensional frequency parameter also known as the Womersley number. The angular frequency is represented by ω. Viscosity and density of the fluid is represented by μ and ρ. The radius of the vessel is R, the pressure gradient magnitude is Pₛ. Bessel function of first kind and order zero is shown as J₀, and i represents the imaginary unit. However, the blood vessels are not rigid and the Navier-Stokes equation can be solved for pulsatile flow in an elastic tube to give:

$$u ( x , r , t ) = Real \{ \frac{{i•P}_{s}}{ρ•ω} ( 1 - G• \frac{{J}_{0} ( {W•i}^{\frac{3}{2} • \frac{r}{R}} )}{{J}_{0} ( {W•i}^{\frac{3}{2}} )} ) ) {• e}^{\frac{iw ( t - x )}{c}} \} \qquad ( 7 )$$

Here c is the wave speed and G is a group parameter function of Poisson's ratio, Young's modulus, diameter of tube and wall thickness of the tube. Although in some physiological conditions the assumption of Newtonian fluid is not valid; the blood flow is still governed by the Cauchy momentum equation.

In fluid mechanics, optical methods of flow visualization such as Particle Image Velocimetry, Laser Speckle Velocimetry and Background Oriented Schlieren are used to obtain better understanding of the flow field. These methodologies are based on comparing two snapshots of the flow field in different times. For example, in laser speckle velocimetry, two snapshots of the speckle patterns are taken, rather than using a single blurred image, and the displacement vectors of the speckle pattern between the two frames are calculated. Finding displacement vectors from a pair of images is commonly referred as optical flow estimation which could be defined as the apparent motion of 2D projection of images between time steps. Cross-correlation methods are the most common methods used in computing displacement between a pair of images. In cross-correlation the whole image is divided into small windows for analysis over which the velocity can be assumed to be constant. Cross-correlation is a conventional method that is used in various applications including particle image velocimetry and laser speckle velocimetry. However, cross-correlation algorithms cause the analysis of speckle patterns to become much more complicated compared to speckle contrast techniques, in which speckle contrast is measured over a single image. Moreover, cross-correlation methods reduce the spatial resolution of the image as well. Because of these disadvantages, speckle contrast imaging has been more popular.

With the recent developments in the computer vision especially in fields of optical flow estimation over the past two decades, more advanced and more accurate method has been developed. Optical flow algorithms were compared with cross-correlation algorithm using a synthetic dataset of noise backgrounds. It was found that optical flow algorithms significantly increase the resolution of displacement calculations.

In this communication, a fluid mechanic approach is taken to visualize blood flow. Thus, different physical behavior of the blood flow, such as the pulsatile behavior as shown in Eqs. (6) and (7), is used to visualize the blood flow. This was achieved by calculating apparent displacement of laser speckle patterns. As discussed earlier, when calculating displacement between a pair of images, optical flow algorithms have better resolution. Hence, optical flow algorithms were deployed to calculate the apparent displacement of image pixels. The differences in optical displacement were used to map the blood vessels. This methodology, hereafter referred to as the Laser Speckle Optical Flow Imaging (LSOFI), is similar to the laser speckle velocimetry and background oriented Schlieren. In the next section the optical flow algorithms are reviewed in sufficient details followed by the section on the image acquisition. Sample results are given in Section 4 followed by conclusions in Section 5.

### Optical Flow Algorithms

The early concept of optical flow algorithms arises from James J. Gibson's (1966) work on visual stimulus provided to animals. Having two images I(x,y,t₀) and I(x,y,t₁) optical flow is defined as the 2D vector field describing the apparent motion of each pixel. The apparent motion computation is based on the assumption of brightness conservation, which states that the pixel intensity of the same physical point is identical in both images,

I(x,y,t)=I(x+δx,y+δy,t+δt) (8)

As previously mentioned, when the speckle pattern is generated blood vessels, the flow causes fluctuations, which cause the intensity of the corresponding pixel in the image to change. Based on the assumption of brightness conservation (Eq. (8)), the change of such intensities in a pair of images leads to an apparent motion of pixels. Conservation of brightness principal could be restated such as if a pixel in the picture frame is selected and the pixel is followed between the pair of images, the intensity of the pixel does not change. The material derivative can be used to describe the brightness conservation, caused by the flow, as:

$$\frac{DI}{Dt} = \frac{\partial I}{\partial t} + \frac{δ x}{δt} \cdot \frac{\partial I}{\partial x} + \frac{\partial y}{\partial t} \cdot \frac{\partial I}{\partial y} = \frac{\partial I}{\partial t} + u . \frac{\partial I}{\partial x} + v . \frac{\partial I}{\partial y} = 0 \qquad ( 9 )$$

Eq. (9) states that the apparent motion of pixel is dependent on both spatial and temporal gradient of pixel intensities. For each pixel in Eq. (9), there are two unknowns u and v. Therefore, solving Eq. (9) requires additional constraints, which are discussed next.

### Horn-Schunck Method

In order to solve Eq. (9), Horn and Schunck (1980) introduced another set of constraint known as the smoothness constraint. Based on Horn-Schunck's approach, for slow movements, the displacement is considered smooth when the square of the gradient of velocities is minimum. Their methodology is based on the idea to minimize:

$${\int }_{Ω} {( \frac{DI}{Dt} )}^{2} + α ( \vert ∇ δ x {\vert}^{2} + \vert ∇ δ y {\vert}^{2} ) dxdy \qquad ( 10 )$$

the smoothness constraint. After basic transformations, it is shown that minimization of Eq. (10) is equivalent to minimization of

$${\int }_{Ω} {( \frac{DI}{Dt} )}^{2} + α ( {( ∇ . U )}^{2} + \vert ∇ \times U {\vert}^{2} ) dxdy \qquad ( 11 )$$

where

U=u·î+v·ĵ

is the velocity vector. Minimization of divergence of velocity (∇·U) corresponds to the fact that the flow is incompressible, and minimization of ∇×U signifies that the vorticity, corresponding to the blood flow field between a pair of images, is minimized.

### Lucas-Kanade Method

Lucas-Kanade method assumes that the motion between the two frames is slow and the displacement is constant in each small blocks of the image. Therefore, Eq. (9) can be assumed to hold for all pixel of a window (Lucas and Kanade, 1981). Using the weighted least-square fit and assuming a window function, W, to emphasize the constraint at the center of each window, the Lucas-Kanade method has the following form of solution for velocity components u and v:

$$[ \begin{matrix}u \\ v\end{matrix} ] = {[ \begin{matrix}\sum {{W}^{2} ( \frac{\partial I}{\partial x} )}^{2} & \sum {W}^{2} ( \frac{\partial I}{\partial x} ) ( \frac{\partial I}{\partial y} ) \\ \sum {W}^{2} ( \frac{\partial I}{\partial x} ) ( \frac{\partial I}{\partial y} ) & \sum {{W}^{2} ( \frac{\partial I}{\partial x} )}^{2}\end{matrix} ]}^{- 1} \cdot [ \begin{matrix}- \sum {W}^{2} ( \frac{\partial I}{\partial x} ) \cdot ( \frac{\partial I}{\partial y} ) \\ - \sum {W}^{2} ( \frac{\partial I}{\partial y} ) \cdot ( \frac{\partial I}{\partial t} )\end{matrix} ] \qquad ( 12 )$$

### Farneback Method

The Farneback (2003) method does not solve Eq. (9). Instead it approximates a neighborhood of both frames at a time t1 and t2 using a polynomial function. For the case of quadratic polynomial, the intensity can be written as:

Iₜ_{₁}(x)=x^{T}A₁x+b₁^{T}x+c₁ (13)

New signal can be constructed using a global displacement (d) as

$$\begin{matrix}\begin{matrix}{I}_{{t}_{1}} ( x - d ) = {( x - d )}^{T} {A}_{1} ( x - d ) + {b}_{1}^{T} ( x - d ) + {c}_{1} \\ = {x}^{T} {A}_{1} x + {( b 1 - 2 {A}_{1} d )}^{T} x + {d}^{T} {A}_{1} d - {b}_{1}^{T} d + {c}_{1}\end{matrix} & ( 14 ) \\ {I}_{{t}_{2}} ( x ) = {x}^{T} {A}_{2} x + {b}_{2}^{T} x + c & ( 15 )\end{matrix}$$

Since Iₜ₁(x−d)=Iₜ₂(x), equating the coefficient in the quadratic polynomial yields to b₂=b₁−2A₁d. From b₂=b₁−2A₁d the transition value d could be solved if A1 is non-singular. In principle, Eqs. (14) and (15) can be equated at every pixel and the solution may be obtained iteratively. Farneback (2003) noted that the pointwise solution is too noisy. Instead, the displacement may be assumed to be slow varying and satisfies a neighborhood of values of x. The Farneback algorithm, combines the polynomial approximation with multi scale resolution to produce optical flow results for each pixel of the image.

### Image Acquisition

In order to produce dataset for analysis of LSOFI, Laser speckle patterns were applied to the cranial bone of the mouse. The animal preparation and experimental setup have been explained in detail by Davoodzadeh et al. (2018). The images were obtained by a 12-bit CMOS camera with a rolling shutter (Thorlabs DCC1545). The camera system was attached to a microscope with 10× magnification with focus plane of 0.3 mm below the surface. Laser speckle patterns were generated using a continuous wave laser of 632.8 nm wavelength coupled with beam expanders and diffusers. The resulting image had 1024×1280 pixel resolution acquired with a frame rate of 14.5 Hz. For the purpose of this study, images with the exposure time of 12.5 ms were used. The result of applying LSOFI to the sample dataset is presented next.

### Results and Discussions

First, the spatial resolution of LSOFI was compared to LSCI methods. This section is followed by evaluating effect of sampling time and temporal resolution on LSOFI and LSCI. Thereafter, various filters were applied to the resulting dataset to increase the temporal and spatial resolution and help to identify the blood flow. At last, quantitative values calculated for LSOFI are presented.

### Qualitative Visualization of Blood Flow Using Optical Flow Algorithms

To present the visualization results, a new variable M is introduced as:

$$M = \frac{1}{T} {\int }_{0}^{T} \sqrt{{u ( t )}^{2} + {v ( t )}^{2}} dt \qquad ( 16 )$$

where u(t) and v(t) are horizontal and vertical motions calculated for each pixel in pair of frames and T is averaging time over series of image frames. The frames are averaged primarily to increase the signal to noise ratio.

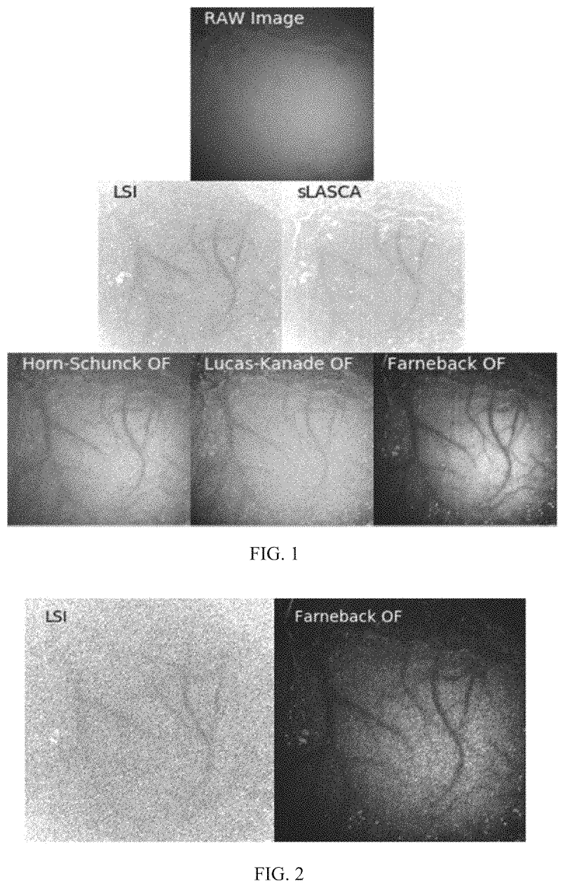

FIG. 1 is visualization of M when calculated using Horn-Schunck, Lucas-Kanade and Farneback optical flow algorithms. FIG. 1 also shows the results of applying LSCI to the image datasets. Laser Speckle Imaging method (LSI) and Spatially Derived Contrast Using Temporal Frame Averaging (sLASCA) were deployed to the dataset to calculate the Laser speckle contrast K. LSI method determines K over T number of frames (LSI image in FIG. 1 uses T=190 frames). sLASCA method determines K by averaging T number of Laser Speckle Contrast Analysis (LASCA) images. In LASCA, K is measured in I image over a pixel window (sLASCA image in FIG. 1 uses T=190 frames and window size of 5×5).

FIG. 1, clearly demonstrates that at the same temporal resolution, Horn-Schunck, Lucas-Kanade, and Farneback optical flow algorithms have higher resolution than LSCI methods. Thus, optical flow algorithms make it easier to identify and visualize the blood vessels. In the LSCI methods, sLASCA has very low spatial resolution compared to the LSI method. Therefore LSI method is compared to optical flow algorithm for this work. Among the optical flow algorithms, Horn-Schunck is more sensitive to smaller displacements. This results in more noise as compared to the Farneback algorithm, which is sensitive to all scales of motion.

FIG. 1. First row is a snapshot of the raw image obtained. The second row is the result of mapping K using LSI and sLASCA algorithms. The third row demonstrates the mapped value of “M” using Horn-Schunck, Lucas Kanade and Farneback optical flow algorithms. It can be seen that sLASCA has very low spatial resolution compared to LSI method. This could be the artifacts of using a rolling shutter camera. The LSOFI algorithms produce higher resolution images compared to LSCI imaging methods.

As shown in Eq. (16), to increase the signal to noise ratio of the resulting images, T number of frames were selected and averaged. When using LSI imaging method, T is the number of frames used for calculation of K. When using sLASCA, T is the number of frames used for averaging the results.

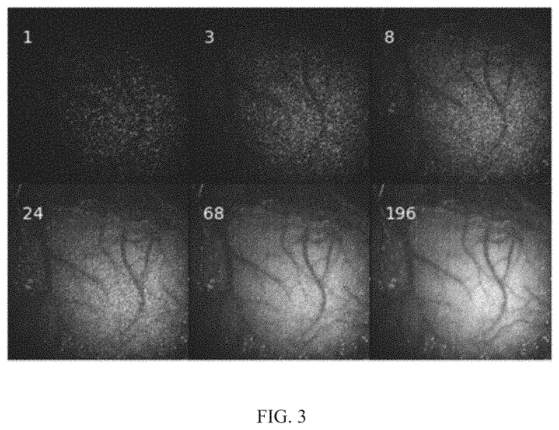

To investigate the effect of averaging over the dataset, the number of frames was reduced to 20 images which corresponds to 1.37 s. Because of higher spatial resolution, Farneback's algorithm was chosen as the representative of the optical flow algorithms, and LSI method was chosen to represent LSCI methods. The results of applying Farneback algorithm and LSI algorithm over 20 raw images are shown in FIG. 2.

FIG. 2 shows that for smaller averaging, Farneback optical flow algorithm has higher resolution compared to LSI. In other words, Farneback optical flow algorithm requires a smaller number of images to produce a reasonable flow visualization as compared to the LSI method. This specification is very helpful towards quasi-real time visualization of blood vessels. Comparing Farneback's results from FIGS. 1 and 2, that the results show that as the averaging time increases, the amount of noise in the image decreases; which leads to clearer blood vessel visualization.

FIG. 3 demonstrates the effect of averaging time on the output of the Farneback's optical flow algorithm. It can be seen from FIG. 3 that when the number of image pairs used for temporal averaging increases, the resolution of the output image increases. One of the optical flow algorithms advantages over LSI is that the optical flow analysis will output resulting image gradually as images are captured. LSI, which uses the temporal properties of speckle patterns, require all the inputs at once to produce the resulting image.

FIG. 2. Image processing results of applying LSI and Farneback optical flow algorithm over 20 frames of raw-images. It can be seen that Farneback optical flow algorithm requires less images to produce a meaningful result.

FIG. 3. Image processing results using Farneback's optical flow algorithm with different averaging times. The number on the top left corner represents the number of pair of images used to process the image. As the number of data frames increases, the resolution of resulting image increases as well. It can be seen that for higher spatial resolution image, more images are required.

### Post Data Processing

### Noise Reduction of the Images

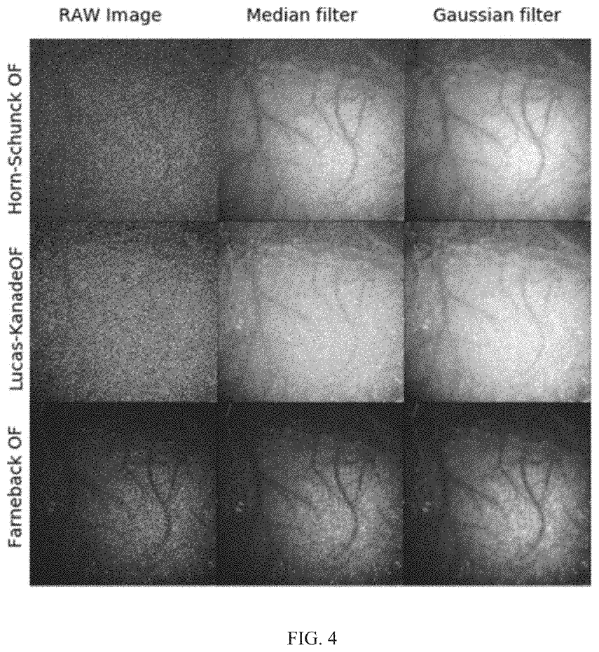

As shown in the previous section, optical flow algorithms, have higher resolution compared to Laser Speckle Contrast Imaging algorithms (LSCI). Moreover, it has been shown that as the number of data samples increases, the resulting image will have less noise and therefore higher resolution. However, it is not always practical to increase the number of data points to reduce the image noise. Hence noise reduction algorithms and filters could be applied to enhance the resolution. FIG. 4 demonstrates the effects of the Gaussian filter and median filter to the outcomes of the optical flow algorithms. FIG. 4 illustrates the result of applying filters and image noise reduction algorithms. For each of the optical flow algorithms, 10 images were used. Applying filters reduces the need for longer averaging time. It can be seen that when applying a filter, Horn-Schunck algorithm produces a higher spatial resolution image. Even though the Farneback's algorithm produces higher spatial resolution image without a filter.

### Image Segmentation and Identification of Blood Vessels

Optical flow's high spatial resolution results make it possible to identify and segment the blood vessels in the image. Via image segmentation algorithms. In computer vision, Image segmentation is used to partition a digital image into different regions. By partitioning digital images into different regions, the overall analysis becomes less computationally expensive. Frangi et al.'s (1998) “multiscale vessel enhancement filter,” is one of the standard image segmentation algorithms, used to identify the blood vessels in a digital image. To identify the blood vessels, Frangi's filter was applied to the results of the optical flow algorithms. The results are shown in FIG. 5.

FIG. 5 demonstrates that the combination of Frangi and Gaussian filtering lead to better identification of blood vessels. The intensity of the image correlates to the amount of vesselness calculated using the Frangi filter. This can later be implemented for automatic blood vessel detection using LSOFI.

### Quantitative Analysis of Laser Speckle Optical Flow Imaging

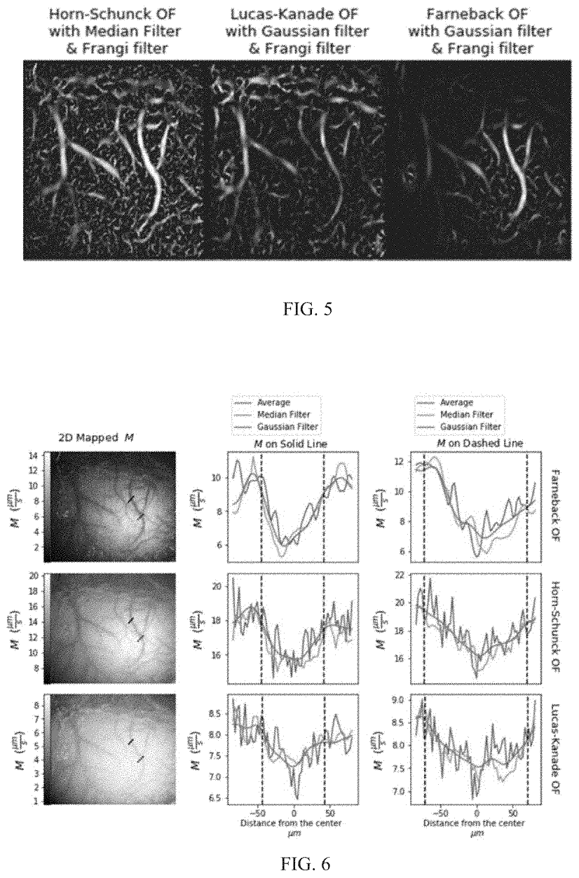

The previous sections demonstrated Laser Speckle Optical Flow imaging advantages over LSCI methods qualitatively. It also has been shown that the better resolution of LSOFI can help in developing an autonomous blood vessel detection system. However, like many LSCI methods, the correlation of quantitative value of each representing pixel with the blood velocity profile is uncertain. The quantitative unitless value of κ is often assumed to have a correlation with the decorrelation time as explained in Eqs. (2)-(5). However, this assumption has numerous issues that prevent LSCI method for becoming a quantitative measurement methodology. In contrast to the LSCI methods, the hypothesis of the LSOFI, introduced in this paper, is physically based on the Cauchy momentum equation. FIG. 6 presents the apparent displacement, M, for different calculation methods. The first column shows the 2D mapped displacement of the apparent motion. The second column represents the apparent motion calculated at the solid line shown in the first column. The third column represents the apparent motion calculated at the dashed line shown in the first column. The vertical dotted line in the second and third column shows the approximate boundary of the vessel at the location of calculation. For the second and third column, the values of averaged displacement over 190 frames were calculated along with averaged displacement over 10 frames filtered using Gaussian and median filters. Each row represents the optical flow algorithm used for calculation. It can be interpreted from FIG. 6 that when Gaussian and median filters are applied to the LSOFI results of 10 frames of images (T=10 in Eq. (16)) have the same spatial resolution as when 190 frames of LSOFI results are averaged (T=190 in Eq. (16)) with no filters. In simpler words, Gaussian and median filter reduce the need for additional frames. FIG. 6 shows that although the magnitudes calculated by the optical flow algorithms differ, the different LSOFI algorithm produce almost the same profiles.

### CONCLUSIONS

Laser speckle contrast imaging is known as a convenient method for visualizing flow in vessels. Common LSCI methods mapped the values of speckle contrast “K” to produce a resulting image. In this work, a new approach of laser speckle imaging, dubbed Laser Speckle Optical Flow Imaging (LSOFI) was introduced. In contrast with LSCI methods which use the statistical properties of speckle patterns for flow visualization, LSOFI maps the values of the apparent displacement of laser speckle patterns. Strictly speaking, the noise caused by the blood flow is governed by Cauchy momentum equation and in some cases Navier-Stokes equation. This is in contrast with the noise displacement in the surrounding tissue which has different governing equation. LSOFI captures the speckle displacement caused by different physical behavior and creates a mapped image. It has been shown that LSOFI has advantages over LSCI methods both in temporal and spatial resolution. In other words, LSOFI can be used to produce higher resolution images compared with LSCI method using less frames. Moreover, the architecture of the LSOFI is optimal for Graphics Processing Unit (GPU) computing platforms such as Nvidia's CUDA platform. Since the GPU computation increases the speed of LSOFI, the GPU enabled LSOFI could be deployed to the embedded systems such as Nvidia's JETSON to create a fast and fully functional quasi-real time blood flow imaging system. Like LSCI methods, the substantial challenge remains if LSOFI could be used as a quantitative tool for assessing blood flow in vessels.

Although the LSOFI results have the same order of magnitude as some reported values for blood flow in mouse's brain vessels, further experiments using precise velocity measurements like LDF is required to validate LSOFI as a quantitative tool for flowmetry. Nonetheless, LSOFI could be used as a valid qualitative tool for blood flow visualization.

FIG. 4. Result of applying a Gaussian and median filter on the optical flow result of 10 pair of images. The row indicates the optical flow algorithm used, and the columns indicate the filter applied. It can be seen that applying Gaussian and median filters to the raw results of Horn-Schunck and Lucas-Kanade optical flow algorithms lead to images with higher resolution.

FIG. 5. Results of applying a Frangi filter to the output of optical flow algorithms. The intensity of each pixel shows the vesselness calculated by Frangi filter. It can be seen for a small number of frames; the Horn-Schunck algorithm visualizes the blood vein better than other optical flow algorithms. However, the results of Farneback optical flow have less noise.

FIG. 6. Quantitative analysis of displacement values. The first column presents 2D mapped values of M. Second and third column represent the calculated apparent motion, M, at the solid and dashed lines marked in the first column, respectively. The vertical dotted line in the second and third column shows the approximate boundary of the vessel at the location of the solid and dashed lines. For the second and third column, the value of averaged displacement is calculated over 190 frames (T=190 in Eq. (16)) along with averaged displacement calculated over 10 frames (T=10 in Eq. (16)) using Gaussian and median filters. Each row represents the optical flow algorithm used for calculating speckle displacement.

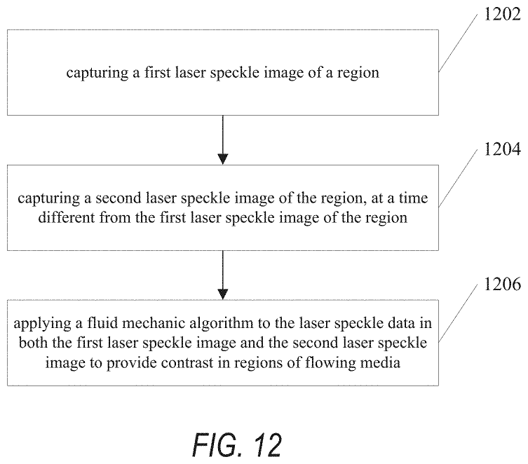

FIG. 12 shows an example method of imaging according to an embodiment of the invention. In operation 1202, a first laser speckle image of a region is captured. In operation 1204, a second laser speckle image of the region is captured, at a time different from the first laser speckle image of the region. In operation 1206, a fluid mechanic algorithm is applied to the laser speckle data in both the first laser speckle image and the second laser speckle image to provide contrast in regions of flowing media.

While a number of advantages of embodiments described herein are listed above, the list is not exhaustive. Other advantages of embodiments described above will be apparent to one of ordinary skill in the art, having read the present disclosure. Although specific embodiments have been illustrated and described herein, it will be appreciated by those of ordinary skill in the art that any arrangement which is calculated to achieve the same purpose may be substituted for the specific embodiment shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and fabrication methods are used. The scope of the invention should be determined with reference to the appended claims, along with the full scope of equivalents to which such claims are entitled.

### ADDITIONAL NOTES

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

In addition, it is to be understood that the phraseology or terminology employed herein, and not otherwise defined, is for the purpose of description only and not of limitation. Any use of section headings is intended to aid reading of the document and is not to be interpreted as limiting, information that is relevant to a section heading may occur within or outside of that particular section.

The term “substantially simultaneously” or “substantially immediately” or “substantially instantaneously” refers to events occurring at approximately the same time. It is contemplated by the inventor that response times can be limited by mechanical, electrical, or chemical processes and systems. Substantially simultaneously, substantially immediately, or substantially instantaneously can include time periods 1 minute or less, 45 seconds or less, 30 seconds or less, 20 seconds or less, 15 seconds or less, 10 seconds or less, 5 seconds or less, 3 seconds or less, 2 seconds or less, 1 second or less, 0.5 seconds or less, or 0.1 seconds or less.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used herein can allow for a degree of variability in a value or range, for example, within 10%, within 5%, or within 1% of a stated value or of a stated limit of a range.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method of imaging a flowing media, comprising:
capturing a first laser speckle image of a region;
capturing a second laser speckle image of the region, at a time different from the first laser speckle image of the region; and
applying a fluid mechanic algorithm to the laser speckle data in both the first laser speckle image and the second laser speckle image to provide contrast in regions of flowing media.

2. The method of claim 1, wherein providing contrast in regions of flowing media includes providing contrast in regions of flowing blood.

3. The method of claim 2, wherein providing contrast in regions of flowing media includes providing contrast in regions that include multiple blood vessels networks within the region.

4. The method of claim 1, wherein applying a fluid mechanic algorithm includes applying a pulsatile flow algorithm.

5. The method of claim 1, wherein applying a fluid mechanic algorithm includes applying a pulsatile flow algorithm in an elastic tube.

6. The method of claim 1, further including calculating a quantitative value of flow in the flowing media.

7. An imaging device, comprising:
at least one processor;
a laser speckle imaging device;
a storage device comprising instructions, which when executed by the at least one processor, configure the at least one processor to operate the laser speckle imaging device, and perform operations comprising:
capturing a first laser speckle image of a region;
capturing a second laser speckle image of the region, at a time different from the first laser speckle image of the region; and
applying a fluid mechanic algorithm to the laser speckle data in both the first laser speckle image and the second laser speckle image to provide contrast in regions of flowing media.

8. The imaging device of claim 7, wherein the instructions are configured to provide contrast in regions of flowing blood.

9. The imaging device of claim 8, wherein the instructions are configured to provide contrast in regions that include multiple blood vessels networks within the region.

10. The imaging device of claim 7, wherein applying a fluid mechanic algorithm includes applying a pulsatile flow algorithm.

11. The imaging device of claim 7, wherein applying a fluid mechanic algorithm includes applying a pulsatile flow algorithm in an elastic tube.

12. A non-transitory computer-readable medium comprising instructions, which when executed by at least one processor, configure the at least one processor to perform operations comprising:
capturing a first laser speckle image of a region;
capturing a second laser speckle image of the region, at a time different from the first laser speckle image of the region; and
applying a fluid mechanic algorithm to the laser speckle data in both the first laser speckle image and the second laser speckle image to provide contrast in regions of flowing media.

13. The non-transitory computer-readable medium of claim 12, wherein the instructions are configured to provide contrast in regions of flowing blood.

14. The non-transitory computer-readable medium of claim 13, wherein the instructions are configured to provide contrast in regions that include multiple blood vessels networks within the region.

15. The non-transitory computer-readable medium of claim 12, wherein applying a fluid mechanic algorithm includes applying a pulsatile flow algorithm.

16. The non-transitory computer-readable medium of claim 12, wherein applying a fluid mechanic algorithm includes applying a pulsatile flow algorithm in an elastic tube.
